(19) United States
(12) Patent Application Publication
(10) Pub. No.: US 2003/0032870 A1
(43) Pub. Date: Feb. 13, 2003

(54) Method for psychophysiological detection of deception through brain function analysis

(76) Inventor: Lawrence A. Farwell, Fairfield, IA (US)

Correspondence Address: KILE GOEKJIAN LERNER & REED PLLC, THE EVENING STAR BUILDING, 1101 PENNSYLVANIA AVE, N.W., SUITE 800, WASHINGTON, DC, 20004, US

(21) Appl. No.: 10/213,089

(22) Filed: Aug. 7, 2002

Related U.S. Application Data
(60) Provisional application No. 60/310,246, filed on Aug. 7, 2001.

(51) Int. Cl. A61B 5/00

(52) U.S. Cl. 600/300

(57) ABSTRACT

The subject invention comprises a method whereby a deceptive individual will be required to perform a specific cognitive task in order to accomplish deception, which differs from a cognitive task that is performed by a truthful individual in response to the same instructions. The psychophysiological manifestations of the cognitive task or of the increased cognitive activity involved in the task are measured. The brain waves or other psychophysiological data are then analyzed to distinguish the types or levels of cognitive activity produced by the cognitive task for truthful and deceptive individuals.

### RELATED PATENTS

This application claims the benefit of U.S. Provisional Application No. 60/310,246, filed Aug. 7, 2001 and relates to prior U.S. Pat. Nos. 5,363,858 entitled “Method and Apparatus for Multifaceted Electroencephalographic Response Analysis (MERA);” 5,406,956 entitled “Method and Apparatus for Truth Detection;” and 5,467,777 entitled “Method for Electroencephalographic Information Detection;” all of common inventorship with the subject application. The disclosures of these prior patents are hereby incorporated by reference.

### BACKGROUND OF THE INVENTION

### FIELD OF THE INVENTION

The present invention relates to a method for psychophysiological detection of deception through brain function analysis.

Brain Fingerprinting with the Farwell MERMER System: An Effective Brain-Wave Technology Outside the Realm of Detection of Deception

New technologies have been developed recently to use brain waves in forensic science applications outside the realm of detection of deception. Dr. Lawrence Farwell invented the technique of Brain Fingerprinting, also known as the Farwell MERMER System (for Memory and Encoding Related Multifaceted Electroencephalographic Response), and the Farwell MERA System (for Multifaceted Electroencephalographic Response Analysis). This system is described in the above referenced three patents. This new technology uses brain waves to detect the presence or absence of information stored in the brain—including crime-relevant information that can uniquely identify a perpetrator. In both research and field applications, Brain Fingerprinting has been accurate in detecting information in the brain.

Dr. Farwell and his colleagues used Brain Fingerprinting to identify with a high degree of accuracy which individuals in a group were FBI agents and which were not by measuring brain responses to words and phrases that only an FBI agent would recognize which were presented on a computer screen. Similarly, Dr. Farwell used Brain Fingerprinting to identify serial killer J. B. Grinder as the murderer of Julie Helton by measuring Grinder's brain-wave responses to stimuli relevant to that crime. Brain Fingerprinting also was accurate in over 100 tests conducted by Dr. Farwell on contract with the CIA.

Although Brain Fingerprinting has been shown to be a highly accurate means of identifying criminals or individuals associated with a particular group, Brain Fingerprinting can only detect whether or not a person has participated in a crime or other activity under investigation. It is not designed to determine whether or not the person is lying about that crime or situation. In other words, Brain Fingerprinting is not a method of detection of deception. This invention focuses specifically on the use of brain waves and other psychophysiological measurements in detection of deception or credibility assessment.

Conventional Polygraphy

Psychophysiological detection of deception has conventionally involved the measurement of physiological processes mediated by the autonomic nervous system (ANS), such as skin conductance (related to sweat gland activity), cardiovascular activity, and breathing. The basic theory behind this practice, commonly known as lie detection or polygraphy, is that when an individual is lying he is likely to be more emotionally aroused than when he is telling the truth, and this emotional arousal causes a physiological state of arousal that can be measured.

Conventional polygraphy is described in detail in the above referenced U.S. Pat. No. 5,406,956. There has been considerable interest recently in developing alternatives which detect deception or assess credibility through measuring central nervous system activity as evidenced by brain waves, or through other methods that are different from conventional polygraphy.

Types of Electroencephalographic Measurements

Electroencephalography (EEG) involves non-invasive measurement at the scalp of electrical activity generated by the brain. EEG is discussed in detail in the three patents referenced above. EEG measurements are of basically two kinds, event-related potentials (ERPs) and ongoing EEG.

Ongoing EEG

Ongoing electrical brain activity is measured non-invasively from the scalp with sensors and an electroencephalographic amplifier. Electroencephalograph (EEG) data can be analyzed by computer. EEG signals are measured and analyzed over a period of several seconds to several minutes, and in some cases even hours. Ongoing EEG primarily provides information regarding the processing that takes place in the brain over a span of time in excess of a second or two. In contrast to event-related potentials (see below), the processes measured by ongoing EEG are not ordinarily associated with the short-term processing of discrete stimuli, but rather with ongoing brain processes, including complex mental, intellectual, verbal, and creative activities.

Event-related Potentials

Event-related potentials (ERPs) measure short-term electrophysiological events. Event-related potentials are short-term changes in electrical voltage “potential” measured from the scalp that are “related” to an “event.” The event is a particular stimulus and the subject's processing of that stimulus. The event related potential is a manifestation of the sensory or cognitive processing elicited by that stimulus. ERPs range in latency from a few milliseconds to a couple of seconds following the stimulus that elicits them. In some cases where a warning stimulus informs the subject of the imminent arrival of another anticipated stimulus, the event-related potential may precede the second stimulus and manifest preparatory activity for the anticipated stimulus or the subject's anticipated response to it. The stimulus is repeated many times, and the electrical brain responses time-locked to the stimulus are averaged to produce event-related potential measure. In any case, event-related potentials take place over a short period of time, and are related to a stimulus that occurs at a specific point in time. They are an index of brief, short-term sensory or cognitive processes that take place on a scale of a couple of seconds or less.

Event-related potentials play a major role in the invention described in the above referenced U.S. Pat. No. 5,406,956. As discussed above, this technology detects information, and has nothing to do with detecting truthfulness, deception, or credibility. ERPs are suited for detecting information relevant to particular, specific, discrete stimuli—for example, the details of a crime that would be known only to the perpetrator—which may shed light on what crimes or other actions have been perpetrated by a specific individual.

Processes Revealed by Central Nervous System Measurements

Since the brain is intimately involved in communication, it is in principle possible to detect deception or assess credibility using central nervous system measurements, that is, to use measurements of brain activity such as brain waves in lie detection.

Central nervous system measurements can, in principle, reveal two different kinds of brain processes: emotion and cognition.

In order to be an effective means of detection of deception, brain wave measurement must reveal a significant and clearly distinguishable difference in brain activity when a subject is lying versus telling the truth. To accomplish this goal, we must discern either an emotional difference or a cognitive difference.

Difficulties with the Use of Brain Waves in Detection of Deception

1. Emotion processes: No accurate brain-wave indicators; Susceptibility to countermeasures; Unpredictability

There are several difficulties inherent in attempting to use brain waves to detect emotional differences associated with lying. To begin with, there are no known techniques for using brain waves to distinguish accurately between different emotions. Even if we did develop a technique that could distinguish between different emotions with extremely high accuracy, emotions are not necessarily a reliable indicator of truthfulness or deception. Emotions can be manipulated quite readily through mental countermeasures. Moreover, the emotions actually elicited by an interrogatory process may not be the emotions that are intended by the interrogator and that are needed for an accurate assessment of the subject's truthfulness or deception.

One difficulty with conventional, autonomic nervous system-based polygraphy is that individuals can be trained to beat the test. Research has shown that an individual who knows how a polygraph test works (that is, can recognize the irrelevant, relevant, and control questions) can manipulate his emotions so that his emotional and concomitant physiological response is larger to the control than to the relevant questions. This results in a false negative response. Any brain wave test that depends on emotions, no matter how effective the brain-wave measurements are in identifying specific emotions, will inevitably be susceptible to similar difficulties.

Moreover, the emotions that are elicited by the questions asked may not follow the pattern intended by the person who designed or administered the questions. A false positive response can result if an innocent individual's emotional and concomitant physiological response is for some reason larger to the relevant than to the control questions. There is considerable controversy over how much of a problem this is with conventional polygraphy, but in the same way that it is a problem with conventional techniques, it will also inevitably be a problem with any emotion-based brain-wave technique that may be developed.

For these and other reasons, no effective emotion-driven brain-wave technique for detection of deception has been developed, nor can we expect a truly effective technique to be developed in the future.

2. Cognitive measurements: Only trivial cognitive processes elicited by previous methods

Brain-wave responses, particularly event-related potentials, have shown promise in providing an objective means to measure cognitive processes in the laboratory. Event-related potentials are one measurement used in the method and apparatus described in U.S. Pat. No. 5,406,956 to detect information that may be relevant to a crime or other investigated situation. As discussed above, in this prior art, event-related potentials are used to detect information, not lying.

Some attempts have been made to detect lying based on cognitive brain processes and accompanying brain-wave measurements such as event-related potentials, through using a format based on or similar to the questioning format employed in conventional polygraphy. In conventional polygraphy, the subject is asked questions that can be answered by either yes or no. The questions are discussed in detail with the subject before the test. This format has the effect of minimizing cognitive activity (and potential cognitive differences) during the actual test.

Consider, for example, the cognitive processes involved in the following interchange, in which a subject is questioned about a crime he knows about and denies: The following is a relevant question in a conventional polygraph test, followed by the subject's answer.

“Did you shoot John Jones?”

“No”

“No” is the expected answer, whether the suspect is lying (and guilty) or telling the truth (and innocent). The difficulty here, from a cognitive point of view, is that whether the subject is lying or telling the truth, there is extremely little cognitive activity involved in answering the question. To put it in non-technical terms, answering the question is a no-brainer in any case. The subject, whether truthful or not, knows what the question is and knows exactly what his answer will be. He undoubtedly has thought about it extensively before; he may be thinking extensively about this and other things during the interrogation, but the cognitive activity devoted to making this answer is trivial in every case.

Cognitively, there is very little to distinguish one trivial cognitive process from another. It comes as no surprise, then, that any differences there may be between truthful and deceptive subjects performing this and similar cognitively minor tasks involved in responding to yes/no questions and the like have not been shown to be reliably or accurately detectable using brain-wave measurements, particularly measures such as ERPs that are ordinarily used to measure cognitive differences.

For this and other reasons, conventional attempts to detect lying accurately using brain responses elicited by cognitive processes have not been entirely successful in the past. For the same reasons, it is unlikely that similar attempts will be successful in the future.

Shortcomings in Previous Methods for Detection of Deception: The Failure of the Search for a Lie Response

Previous systems for psychophysiological detection of deception have attempted to detect deception or lying, per se, by measuring psychophysiological processes hypothesized to accompany deception. The primary difficulty with this approach is that lying is not a unitary phenomenon (and, in fact, neither is telling the truth). Since there is not a unitary “lie process”, it is not surprising that researchers have found no evidence that there exists a unique “lie response” that can be measured psychophysiologicaily. Some researchers have searched for not one lie response, but a set of responses brought about by a set of cognitive or emotional processes that are hypothesized to be engaged in when one lies. There is no evidence, however, that a unique set of several other cognitive or emotional processes exists that is engaged in whenever one is lying. On the contrary, a consideration of the widely varied conditions, intentions, goals, strategies, and motivations that may occur during lying under various different circumstances reveals that the cognitive and emotional processes that can be engaged in while lying do not constitute a unique set. Thus, searching for multiple emotional or cognitive substrates of a lie response and their psychophysiological manifestations, like searching for the mythical lie response itself, has not resulted in a fully satisfactory technology for detection of deception.

In short, previous attempts at detection of deception have used deception as the independent variable, and have searched for dependent variables that could be used as an indication that deception was taking place. Since deception per se is not a unitary phenomenon, it has not served adequately as an independent variable, and therefore the search for dependent variables that provide a marker for it has not yielded entirely satisfactory results.

Unique Contribution of the Present Invention

The unique contribution of the present invention is that, rather than seeking to measure psychophysiological manifestations of deception or other processes called upon in the course of deception, it creates a situation where a deceptive individual will be required to perform a specific (and generally more difficult) cognitive task in order to accomplish his deception, a task that differs in specific ways from the cognitive task that is performed by a truthful individual in response to the same instructions. The psychophysiological manifestations of the cognitive task, or of the increased cognitive activity involved in performing the task, can then be measured.

The difficulties, limitations and desires suggested in the preceding are not intended to be exhaustive, but rather are among many which demonstrate that prior art methods and systems detection of deception will admit to worthwhile improvement.

### SUMMARY OF THE INVENTION

To achieve at least some of the foregoing objects, the subject invention provides a method for psychophysiological detection of deception through brain function analysis. Psychophysiological detection of deception through brain function analysis utilizes brain waves to detect information processing activity in the brain that differentiates between the performance of assigned mental tasks between truthful and deceptive subjects, and also detects the presence or absence of information stored in the brain.

The subject invention is capable using brain waves to detect deception by utilizing critical cognitive-load tasks and a distinguishing analysis method, which have not been present in the prior art for detection of deception. Measuring the amount of brain wave activity involved in performing critical cognitive-load tasks indicates significant differences between truthful responses and deceptive responses. A distinguishing analysis method analyzes brain waves or some other psychophysiological data that distinguish between the types or levels of cognitive activity produced by the critical cognitive-load task of a subject.

### DRAWING

Other objects and advantages of the present invention will become apparent from the following detailed description of preferred embodiments thereof taken in conjunction with the accompanying drawing, wherein:

FIG. 1 is a block diagram of a system in accordance with the subject invention.

### DETAILED DESCRIPTION

Equipment and Technology

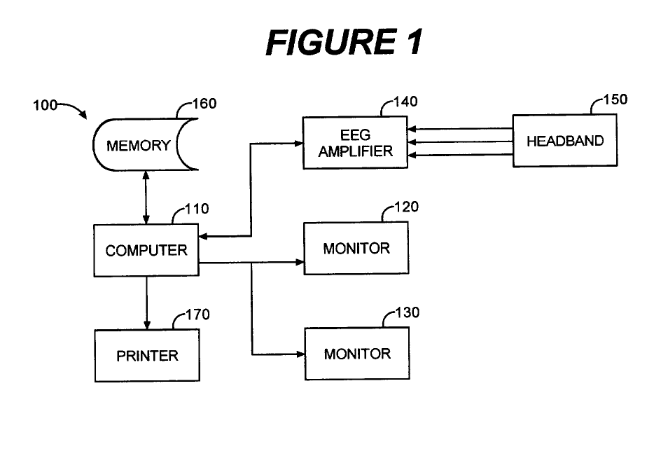

Referring to FIG. 1, a preferred embodiment of the system 100 comprises a personal computer 110 (e.g., Pentium IV, 1 GHz IBM PC); a data acquisition board (e.g., Scientific Solutions Lab Master AD); two monitors 120, 130; a four-channel EEG amplifier system 140 (e.g., Neuroscience); and software for data acquisition and signal processing. The electrodes used to measure electrical brain activity are held in place by a special headband 150 designed and constructed by the inventor for this purpose. The software collects the electroencephalographic and psychophysiological data, and analyzes the data.

In at least one embodiment of the subject invention a monitor 120 is placed before a subject to be tested for deception. The monitor 120 displays information and instructions relevant to a cognitive-load task that the subject is to perform.

During the test for detection of deception, brain electrical activity is recorded from three midline scalp locations on the head: frontal (Fz), central (Cz) and parietal (Pz), referenced to linked ears or linked mastoids (behind the ear). It will be understood that additional brain signals measured from other scalp locations, and other psychophysiological measurements may be used as well. Electrical activity generated by eye movements is recorded by an electrode above one eye. Brain electrical activity is amplified, analog filtered (e.g., low-pass 30 Hz, high pass 0.1 Hz) digitized (e.g., at 333 Hz), analyzed on-line, and stored on a memory device 160.

In addition to displaying the results of the analysis on the monitor 130, the system may also print out on a printer 170 the statistical results, the summary of the textual information, and the waveform displays.

Detection of Deception Using Brain Waves: the Brain Function Analysis System

A. Requirements for an effective brain-wave-based technology for detection of deception

There are two essential ingredients of a successful technology to use brain waves in detection of deception, which have been lacking in previous attempts:

1. A critical cognitive-load task: a task that results in substantial, fundamental, significant differences between the cognitive activity required of a truthful versus a deceptive individual at the time when the brain-wave measurements are being made; and

2. A distinguishing analysis method: A method of analysis of brain waves (or other psychophysiological data) that distinguishes between the two different styles or levels of cognitive activity produced by the critical cognitive-load task for truthful and deceptive subjects.

These two requirements are met by the subject invention.

B. Cognitive activity during interrogation

To understand the task utilized in this invention, it will be instructive to examine the cognitive activity that takes place during an interrogation.

While an innocent suspect is being questioned in a free-form format regarding, say, an espionage crime, his stream-of-consciousness thoughts may go something like this:

“If he asks me about my vacation in Helsinki one more time I think I'll scream. I'm telling him all I can remember, but I've I forgotten a lot of details. Oh, no, now he's onto my trip to New York. I wonder if that waitress named Tanya I dated there was actually from Russia and not Germany as she said. Well, I didn't even tell her where I worked . . .”

The stream-of-consciousness thoughts of a guilty suspect might go something like this:

“I wonder why he keeps asking about Helsinki. Do they know about the papers I gave to Boris? They must know. I'll just deny it again. Maybe they don't know. I shouldn't have been so careless about counter-surveillance. What if I can't convince him? Maybe Tanya in New York told them everything. I never trusted her. I could change my story and say she did it, but then what will she do?”

An innocent, truthful subject spontaneously experiences a stream of thoughts that he could safely reveal to an interrogator. A deceptive subject spontaneously experiences a stream of thoughts at least some of which he could not safely reveal to an interrogator.

C. A critical cognitive-load task that fulfills the first requirement

Consider the following task. During interrogation, the subject is instructed to answer the questions he is asked, and also to report continuously on his stream-of-consciousness thought processes by simply speaking out whatever thoughts come into his mind. For the truthful subject, this is a very simple and easy task. His thoughts and emotions may not be pleasant, but simply speaking whatever thoughts come into his mind as they arise is cognitively an almost trivial task. Since he has nothing to hide, this is the task he will perform.

The deceptive subject has been instructed to perform the same task, but the same instructions result for him in a far more difficult and complex task. Obviously, he cannot simply speak out whatever comes into his mind, because some of the thoughts that come into his mind are about the information he is attempting to hide. He must continuously monitor his thought processes, decide what he can say and what would be incriminating, and make up a plausible, continuous monologue that sounds as if it reflects his spontaneous thoughts when actually it does not. Unlike the truthful subject's task of simply saying whatever spontaneously pops into one's mind, the deceptive subject faces a task requiring considerable mental effort. Cognitively, it is significantly more complex and difficult than the task faced by a truthful subject.

This instruction fulfills the requirement of creating a task that requires markedly and fundamentally different cognitive activity for a truthful subject than for a deceptive subject.

D. Analysis methods for fulfilling the second requirement

The second requirement for an effective technology using brain waves in detection of deception is that we have a viable means to assess these cognitive differences by measuring brain waves. Previous research has uncovered promising methods for accomplishing this goal. Dynamical systems analysis has been shown to be promising in this regard. Furthermore it has been shown that dynamical systems analysis shows promise for detecting differences in cognitive activity elicited by mental tasks. Multifaceted electroencephalographic response analysis or MERA also has proven useful in detecting differences in cognitive activity.

E. Methods for developing comparison data

The psychophysiological measurements taken during the performance of the critical cognitive-load task—when the subject is performing the task that will result in a significant cognitive load if and only if he is deceptive—are compared with other comparison psychophysiological data.

Ideally, comparison data are of two types: 1) high-cognitive-load comparison data: data collected when the subject performing a task that will produce a significant cognitive load for all subjects, whether truthful or deceptive; and 2) low-cognitive-load comparison data: data collected when the subject is not experiencing a significant cognitive load.

The data collected during the cognitive-load task can then be compared with two standards. If the data collected while the subject is performing the critical cognitive-load task are more similar to the data collected during the high-cognitive-load task, this is an indication of deception on the part of the subject. If the critical-cognitive-load data are more similar to the low-cognitive-load data, this is an indication of truthfulness.

Ideally, comparison data will be from the same subject as the test data, although it is also possible to develop population norms to use as comparison data. Comparison data may include any of the following: 1) the subject's own data, taken when he is constrained to perform the cognitive-load task or a task involving a high cognitive load; 2) the subject's own data, when the subject is not experiencing a high cognitive load; 3) a set of standards for truthful subjects; 4) a set of standards for deceptive subjects;

Standard population data for the cognitive-load task when a subject is being truthful vs. deceptive could be developed by gathering data on experimental subjects, or on field subjects when ground truth is known or is later discovered.

The subject's own comparison data could be developed by assigning two different tasks designed to produce a high cognitive load and a low cognitive load respectively in all subjects. The high-cognitive-load comparison data could be developed when the subject is instructed to answer questions while generating a stream-of-consciousness report, and is given some constraints that will necessitate generating a false report (e.g., the report must refer to the subject as a French female in Africa, when he is an American male who has never been outside the USA). Another high-cognitive-load comparison task would be for the subject to be instructed to make up and speak out a fictional story, while simultaneously answering questions about known events, either crime-relevant or not.

The low-cognitive-load comparison data could be collected when the subject is attached to the measuring devices, but is not yet being presented with any task, when he/she is speaking truthfully about items where ground truth is known or which have no relevance to the investigated situation, or when he/she is conducting a simple stream-of-consciousness task that has nothing to do with the investigated situation or any other situation that might demand deception on the part of the subject, or when he/she is performing another cognitively easy task such as listening to music.

F. Detection of deception with brain waves through Brain Function Analysis: The current state of the art

The above described cognitive task and mathematical brain-wave analysis techniques can provide information that can assist in assessing the level of truthfulness or credibility displayed by a subject in the course of questioning. Parts of this technology have been covered by previous patents by Farwell that are incorporated herein by reference (U.S. Pat. Nos. 5,363,858; 5,406,956).

Previous attempts by others to use brain waves in detection of deception have not resulted in a viable technology, and we have seen above that there are substantial scientific reasons for this shortcoming.

Alternative Embodiments of the Technology

For the technology to be effective in providing useful information regarding detecting deception or detecting the level of truthfulness or credibility of a subject, two elements must be present: 1) a substantial task that requires significant cognitive activity of the subject if and only if he is deceptive; and 2) a means of assessing the level of cognitive activity through psychophysiological measures.

In the preferred embodiment the assigned task is to report on one's spontaneous thoughts during interrogation regarding an investigated situation. The means of assessing the level of cognitive activity or effort is measurement of ongoing electroencephalographic activity. Several other alternatives are available for the task and for the assessment method.

A. Alternative cognitive-load tasks

The task described above produces a situation where a non-truthful subject will be required to undertake a substantially more difficult and complicated cognitive task than a truthful subject, in response to the same external task demands. Other tasks may be employed to produce this effect.

Note that lying, per se, is not necessarily more difficult than telling the truth. It depends on the circumstances. Telling a complicated lie is generally more difficult than telling a simple truth. Answering a simple yes/no question truthfully, however, may be more difficult than speaking a one-word lie, particularly when there are significant negative consequences for telling the truth (e.g., exposure and punishment for a crime). In the present invention, we are not depending on the untenable hypothesis that lying is always more difficult than telling the truth. We are creating a situation where a deceptive individual will be forced to employ more cognitive resources to perform a more difficult cognitive task than a truthful individual, in response to the same task instructions.

Although ordinarily it is not significantly more difficult, from a cognitive standpoint, to lie than to tell the truth, specific task instructions can create a situation where a deceptive individual must perform a cognitively more demanding task than a truthful individual, in response to the same instructions. Any task instructions that would allow a truthful individual to report a relatively simple or straightforward truth, but would require a non-truthful individual to think in more complex ways, could meet the requirement.

For example, an individual being questioned about a specific crime could be asked a series of specific questions about his alibi, questions that would require complicated cognitive activity to develop false responses. He could be presented with contradictions between known facts and his statements, and burdened with the difficult cognitive task of inventing new explanations for these contradictions. He could be presented with contradictions between the statements of others and his own statements, between his statements and known facts, or between his statements and his own previous statements. He could be presented with the task of reporting on what he knew about a complicated set of interrelated events, or the interrelated activities of several persons. The truthful individual would have the simple task of simply stating what he knew of the events and people in question. The deceptive individual would have the more difficult and cognitively demanding task of developing plausible lies, while maintaining consistency with previous lies and known facts.

B. Alternative tasks to elicit comparison data

The task used to elicit the comparison data can be of several types. The critical requirement is that the task produce a significant cognitive load for the subject.

The comparison task could be a task unrelated to the investigation and to the cognitive-load task, such as a task involving difficult mathematical computations.

Another alternative is to require the subject to provide a stream-of-consciousness report of his thoughts in a situation where he can be expected to generate a false stream-of-consciousness report due to his need to be deceptive regarding events where ground truth is known. For example, the suspect in one crime could be questioned about other crimes that he is known to have committed but which he would be expected to deny, and the stream-of-consciousness task could be assigned during that questioning. This alternative is available, of course, only in the limited circumstances where there are known subjects about which the subject can be expected to lie.

C. Alternative psychophysiological measurements

In addition to, or instead of, ongoing EEG activity, other psychophysiological measurements may be employed to assess the level of cognitive activity elicited by the task as a means of assessing the level of truthfulness of the subject. Several other psychophysiological measurements are known to be related to cognitive activity.

Cognitive brain activity can be assessed through measuring magnetic fields around the head (as contrasted with the electric fields that are measured by EEG); through positron emission tomography (PET); potentially through magnetic resonance imaging (MRI); through various methods to assess blood flow in the brain, including visible light and laser light.

Cardiac activity, as measured electrophysiologically (electrocardiogram, EKG), can provide information on cognitive activity. Potentially useful parameters include heart rate, heart rate variability, cardiac-sinus arrhythmia, the variations in heart rate as a function of breathing activity, variations in the shape of the EKG signal, variations in the relative and absolute amplitude and timing of the components of the EKG signal.

Muscle activity, as measured electrophysiologically, particularly the activity of muscles in the face and neck, can also shed light on cognitive activity.

Breathing activity, alone or in conjunction with heart rate, can provide information relevant to the level of cognitive activity being undertaken.

Electrodermal activity is also influenced by cognition. Since it is also very much influenced by emotions, it is unlikely to be a reliable measure of cognitive activity when taken alone. In conjunction with other psychophysiological measures, however, electrodermal activity can contribute to a more complete picture of cognitive activity.

D. Other alternative embodiments

An alternative way of assessing the cognitive load required by the critical cognitive-load task, and thereby assessing the differences in cognitive load in truthful and deceptive subjects, is to assign a secondary task to be conducted simultaneously with the critical cognitive-load task (and the questioning, if it is separate from the critical cognitive-load task). When a secondary task is assigned that competes for cognitive resources with the primary task (i.e., the critical cognitive-load task), then the psychophysiological responses or task performance to the secondary task can provide a measure of the cognitive load of the primary task. The more cognitive resources required by the primary task, the less resources are available for the secondary task.

For example, while performing the critical cognitive-load task, a subject is assigned a simple classification task involving classifying and responding to stimuli presented visually on a computer screen or auditorially through headphones. One way of measuring the subject's task-performance responses is to require button presses providing input to a computer. For example, the task could be to push the left button in response to high tones, and the right button in response to low tones.

Psychophysiological responses to the stimuli, such as event-related potentials, are measured. The amplitude, and, in some circumstances, latency, of brain responses to these stimuli can provide a measure of the cognitive resources available for this secondary task. Brain responses include, for example, event related potentials (ERPs) and multifaceted electroencephalographic responses (MERs). The brain responses to the secondary task provide a measure of the cognitive resources that are left over from performance of the primary, cognitiveload task, and thus provide an indirect measure of the resources required by the cognitive-load task. As the difficulty of the cognitive-load task increases, the amplitude of the brain responses to the secondary task decreases. In some cases, latency also increases.

Secondary task performance, for example, reaction time and accuracy, also degrades as primary, cognitive-load task difficulty increases.

In such a situation, a deceptive subject would be performing a more difficult critical cognitive-load task than a truthful subject. Thus, a deceptive subject would experience a greater degradation of secondary-task performance and secondary-task brain responses during the critical cognitive-load task than a truthful subject.

In this alternative embodiment, comparison cognitive-load tasks could be employed as in the preferred embodiment.

In the preferred embodiment, the subject's responses are verbal responses using multiple words. In an alternative embodiment, the subject's responses are one-word responses, yes/no responses, binary responses, or simple responses produced manually with a computer input device such as a mouse or button box. The critical feature here is that the subject must be required to perform a specific cognitive task—not just lying per se—that will be more cognitively demanding for a deceptive than for a truthful subject. As discussed above, lying is not necessarily more difficult than telling the truth, and in some circumstances may be easier. A task may be assigned, or a question or line of questioning may be designed, however, that will result in an greater cognitive load for a deceptive subject than for an innocent subject, even if the required overt responses are simple.

The stimuli eliciting these responses may also be simple, e.g., words flashed on a computer screen, provided that they are presented in the context of a task where responding to them requires significant cognitive activity at that specific time. Such a design has the advantage of being amenable to measurement of short-term responses such as event-related potentials (ERPs) and multifaceted electroencephalographic responses (MERs).

The embodiments described above involve instructing the subjects in such a way that following the instructions would cause a deceptive subject to perform a more difficult cognitive task than the cognitive task performed by a truthful subject, in response to the same instructions. For distinguishing between a truthful subject and a deceptive subject, however, it is actually not even necessary that the task performed by the deceptive subject should be more difficult than the task performed by the truthful subject, only that the tasks must be substantially different for the two types of subjects. As discussed above, simply postulating that deception is different from telling the truth and searching for concomitant psychophysiological differences, as has been attempted extensively in the past, is not an adequate method to reliably detect deception. The task instructions must be designed so as to produce substantial, predictable cognitive differences in the different tasks performed respectively by deceptive and truthful subjects.

Relying on inherent differences between lying per se and telling the truth is inadequate, because, as discussed above, neither lying nor telling the truth is a unitary phenomenon, and there is no evidence that either carries a unique psychophysiological signature. By contrast, a method of eliciting information from a subject that demands the performance of specific, different cognitive tasks from deceptive as contrasted with truthful subjects, combined with a method to detect the different psychophysiological manifestations of the different tasks, can provide an effective means of detection of deception. Such a method is embodied in the following steps: 1. Creating a set of task instructions to be followed during the course of questioning—or a specific line of questioning—that inherently demands the performance of significantly different cognitive tasks by deceptive and truthful subjects in responding; 2. Measuring the psychophysiological manifestations of the cognitive tasks elicited thereby; and 3. Analyzing the psychophysiological responses to determine whether the subject is performing the cognitive task characteristic of a deceptive subject in response to these specific task demands.

Another alternative embodiment involves presenting a line of questioning or task designed to elicit different types of lies and detecting the difference between different types of lies, based on the different cognitive tasks demanded thereby and the different psychophysiological manifestations of these different cognitive tasks. Take, for example, the situation of an individual who is being interrogated and is lying about a crime he has committed. Under such circumstances the liar will typically have a known, rehearsed lie prepared in response to the basic questions about the event, e.g., “Where were you on the night of July 23?”

In this situation, a the cognitive task undertaken in response to such basic questions by a deceptive person win be quite similar to the cognitive task undertaken by a truthful person in response to the same questions. In both cases, they win search their memory and report the contents thereof. The truthful person will search for and report his memory of the event in question, and the deceptive person will search for and report his memory of the rehearsed lie. This similarity of cognitive tasks makes distinguishing between the two difficult.

When, on the other hand, the line of questioning becomes increasingly detailed or complex, or diverges from the central issue at hand, eventually the point will be reached where the deceptive subject win no longer have a rehearsed he prepared in advance. The truthful subject can continue to perform the same task of searching his memory for the answer and reporting the contents of memory. The deceptive subject, on the other hand, must now resort to a different cognitive task, that of making up the information to communicate in response to questioning. This provides an opportunity to conduct brain measurements sensitive to differences in cognitive processing and thereby to detect the different cognitive processing undertaken by truthful versus deceptive subjects. This provides a method to identify the deceptive subject as such.

In this case, the method involves distinguishing not between any truthful statement and any lie, but between a statement that involves reporting on the contents of memory and a statement that involves making up new information. A rehearsed lie, that is, a lie that the individual has planned in advance (but not necessarily told previously), will not involve the cognitive task of making up new information on the spot. An unrehearsed lie will involve this cognitive task. Thus, psychophysiological measurements sensitive to cognitive differences could distinguish the unrehearsed lie from statements that do not involve this cognitive process. Since only a deceptive subject, and not a truthful subject, will tell an unrehearsed lie, this will provide a method of identifying the deceptive subject as such.

Emotional Differences Not Relevant

In addition to cognitive differences, a non-truthful individual might (or might not) experience different emotions during this procedure than a truthful individual. These emotional differences and their physiological manifestations, however, are not what is being assessed by this technology. Although some of the same psychophysiological sensors may be used, the specific patterns of psychophysiological activity detected and analyzed here are designed to reveal differences in cognitive load being experienced by the subject. These differences are brought about by the subject's task performance in response to task instructions specifically designed to require a different and more demanding cognitive task in a deceptive subject from the task performed by a truthful subject.

Applications of the Invention

This invention provides an interrogator with information on the brain activity and concomitant mental processes of a subject of interrogation that are not apparent from simply questioning the subject and assessing verbal and visual cues. The invention provides information relevant to the level of credibility of subjects who are being questioned for any purpose. The invention can be applied to crime suspects, alleged witnesses, and alleged victims. It can also be applied in screening applications, e.g., for security clearances. In addition to providing information bearing on the credibility of a person in a given situation, the invention can be used to guide an interrogator towards specific subject areas where the subject shows evidence of having difficulty maintaining a credible account.

### SUMMARY OF MAJOR ADVANTAGES OF THE INVENTION

After reading and understanding the foregoing description of preferred embodiments of the invention, in conjunction with the illustrative drawing, it will be appreciated that several distinct advantages of the subject method for psychophysiological detection of deception through brain function analysis are obtained.

One advantage of the present invention is that it provides information on the brain activity and concomitant mental processes of a subject of interrogation that are not apparent from simply questioning the subject and assessing verbal and visual cues.

Another advantage of the present invention is that it provides information relevant to the level of credibility of subjects who are being questioned for any purpose.

Yet another advantage of the present invention is that it can be applied to crime suspects, alleged witnesses, and alleged victims for purposes of credibility.

Yet another advantage of the present invention is that it can be applied in screening applications, e.g., for security clearances.

A further advantage of the present invention is that it can be used to guide an interrogator towards specific subject areas where the subject shows evidence of having difficulty maintaining a credible account.

In accordance with the foregoing, the present invention provides a method for psychophysiological detection of deception through brain function analysis.

In describing the invention, reference has been made to preferred embodiments and illustrative advantages of the invention. Those skilled in the art, however, and familiar with the instant disclosure of the subject invention, may recognize additions, deletions, modifications, substitutions and other changes that fall within the purview of the subject invention.

### Other Publications

The disclosures of the following publications are incorporated by reference into the specification.

Farwell, L. A. (1994). U.S. Pat. No. 5,363,858: Method and Apparatus for Multifaceted Electroencephalographic Response Analysis (MERA)

Farwell, L. A. (1995a). U.S. Pat. No. 5,406,956: Method and Apparatus for Truth Detection.

Farwell, L. A. (1995b). U.S. Pat. No. 5,467,777: Method for Electroencephalographic Information Detection.

Farwell, L. A. and Smith, S. S. (2001) Using Brain MERMER Testing to Detect Knowledge Despite Efforts to Conceal. Journal of Forensic Sciences, 46, 1, 135-143.

Rapp, P. E., Albano, A. M., Schmah, T. I., and Farwell, L. A. (1993). Filtered Noise Can Mimic Low Dimensional Chaotic Attractors. Physical Review E, 47,4, 2289-2297.

What is claimed:

1. A method of detecting deception comprising the following steps:
causing a subject to engage in a critical cognitive-load task that is designed to be cognitively demanding if a subject is not truthful, and less cognitively demanding if a subject is truthful;
conducting psychophysiological measurements on the subject;
analyzing the psychophysiological data to assess the level of cognitive activity undertaken by the subject in performing the critical cognitive-load task; and
using said level of cognitive activity as a means to detect deception.

2. A method according to claim 1 wherein the critical cognitive-load task comprises verbally reporting continuously on one's spontaneous thought processes during the course of questioning.

3. A method according to claim 1 wherein the critical cognitive-load task includes a verbal report given in at least one of the following ways:
orally through speech;
through a keyboard to a computer;
through a hand-held input device to a computer; and
through a manually operated speech synthesizer.

4. A method according to claim 1 wherein said step of analyzing psychophysiological data includes comparing the data obtained during the critical cognitive-load task with data from the same subject when the subject is engaging in a cognitive task that is more cognitively demanding than said critical cognitive load task.

5. A method according to claim 1 wherein said step of analyzing psychophysiological data includes comparing the data obtained during the critical cognitive-load task with data from the same subject when the subject is not engaging in a cognitively demanding task as great as the critical cognitive load task.

6. A method according to claim 1 wherein said step of analyzing psychophysiological data includes comparing the data obtained during the critical cognitive-load task with data from the same subject when the subject is engaging in a task that is a lower cognitive load on the subject than the critical cognitive load task.

7. A method according to claim 1 wherein said step of analyzing psychophysiological data includes comparing data obtained during the critical cognitive-load task with data from the same subject when the subject is engaging in a cognitive task that is more cognitively demanding than the critical cognitive load task and at least one of the following steps:
comparing the data obtained during the critical cognitive-load task with data from the same subject when the subject is not engaging in a cognitively demanding task as great as the critical cognitive load task; and
comparing the data obtained during the critical cognitive-load task with data from the same subject when the subject is engaging in a task that produces a cognitive load on the subject lower than the critical cognitive load task.

8. A method according to claim 1 wherein said step of analyzing the critical cognitive-load task includes responding, using verbal responses with multiple words, to questioning regarding a topic to which the subject's truthfulness is being assessed.

9. A method according to claim 8 wherein said questions are unanticipated by the subject.

10. A method according to claim 8 wherein the subject is asked a series of question in quick succession.

11. A method according to claim 1 wherein said critical cognitive-load task includes the subject verbally explaining known facts about a topic to which the subject's truthfulness is being assessed.

12. A method according to claim 1 wherein said psychophysiological measurements conducted include measurements of central nervous system activity.

13. A method according to claim 12 wherein said psychophysiological measurements conducted include electroencephalography (EEG).

14. A method according to claim 12 wherein said analysis of the psychophysiological measurements includes tomography.

15. A method according to claim 12 wherein said psychophysiological measurements conducted include measurements of magnetic fields.

16. A method according to claim 12 wherein said psychophysiological measurements conducted include positron emission tomography.

17. A method according to claim 12 wherein said psychophysiological measurements conducted include magnetic resonance imaging (MRI).

18. A method according to claim 12 wherein said psychophysiological measurements conducted include measurements that assess blood flow in different areas of the brain.

19. A method according to claim 1 wherein said psychophysiological measurements conducted include cardiovascular measurements.

20. A method according to claim 19 wherein said psychophysiological measurements conducted include electrocardiogram (EKG).

21. A method according to claim 19 wherein said psychophysiological measurements conducted include blood pressure.

22. A method according to claim 1 wherein said psychophysiological measurements conducted include electrodermal activity.

23. A method according to claim 1 wherein said psychophysiological measurements conducted include measurements of breathing activity.

24. A method according to claim 19 wherein said analysis of psychophysiological measurements conducted includes both cardiac and breathing activity and the relationship between the two.

25. A method according to claim 12 wherein said psychophysiological measurements conducted include both central nervous system measurements and at least one of the following:
cardiac activity;
electrodermal activity; and
breathing activity.

26. A method according to claim 25 wherein the data from the central nervous system measurements are combined in the analysis with data from at least one of the following:
cardiac activity;
electrodermal activity; and
breathing activity.

27. A method according to claim 4 wherein said step of comparing requires the subject to answer questions while generating a stream-of-consciousness report, and the subject is given constraints that will necessitate generating a false report.

28. A method according to claim 4 wherein said step of comparing includes making up and speaking a fictional story, while simultaneously answering questions about known events.

29. A method according to claim 8 wherein said step of comparing includes responding to questions regarding contradictions between the subject's statements and known facts.

30. A method according to claim 8 wherein said step of comparing includes responding to questions requiring complicated or extensive responses regarding the subject's specific actions in particular situations relevant to a relevant topic.

31. A method according to claim 8 wherein said step of comparing includes responding to questions requiring the description of complicated, interrelated events.

32. A method according to claim 8 wherein said step of comparing includes responding to questions regarding the interrelated activities of at least one other person other than the subject.

33. A method according to claim 8 wherein said step of comparing includes responding to questions regarding what at least one other person has said regarding a relevant topic.

34. A method according to claim 33 wherein said step of comparing further includes at least one contradiction between what the subject has said and what another person has said.

35. A method according to claim 8 wherein said step of comparing includes responding to questions regarding information relevant to a relevant topic that the subject is told during the course of questioning, and that the subject may not have known previously.

36. A method according to claim 8 wherein said step of comparing includes requiring the subject to respond to questions regarding discrepancies between his statements and known facts.

37. A method according to claim 8 wherein said step of comparing includes requiring the subject to respond to questions regarding discrepancies between subject's statements and subject's statements made at a different time.

38. A method according to claim 8 wherein said step of comparing includes responding to questions that are presented in a sequence that is: (a) unknown in advance to the subject; (b) not an easily predictable sequence of topics; and (b) not a chronological sequence.

39. A method according to claim 8 wherein said step of comparing includes maintaining consistency with previous statements by the subject that may not be truthful.

40. A method according to claim 6 wherein said step of comparing includes truthfully answering questions where the truth is known, using multiple-word answers.

41. A method for detecting of deception comprising the following steps:
questioning a subject;
assigning a critical cognitive-load task to be performed during questioning;
assigning a secondary task involving responding to a stimuli to be performed simultaneously with said critical cognitive-load task;
measuring at least one of the following: (a) psychophysiological responses to stimuli presented in the secondary task; and (b) secondary task performance; and
detecting deception based on said measurements.

42. A method according to claim 41 wherein said psychophysiological responses include brain responses.

43. A method according to claim 42 wherein said psychophysiological responses include event-related potentials.

44. A method according to claim 43 wherein said psychophysiological responses include multifaceted electroencephalographic responses.

45. A method according to claim 41 wherein task performance includes at least one of the following: (a) reaction time; (b) accuracy.

46. A method for detecting of deception comprising steps of:
presenting stimuli to a subject, wherein at least some of said stimuli being relevant to a relevant topic, and to which the subject is required to respond;
requiring performance of a task involving responding to said stimuli that is cognitively more demanding for a deceptive subject than for a truthful subject;
measuring at least one of the following: (a) task performance; (b) psychophysiological responses to said stimuli; or (c) psychophysiological responses associated with the subject's overt task-performance responses; and
detecting deception based on said measurements.

47. A method according to claim 46 wherein said stimuli are presented in at least one of the following ways: (a) visually; (b) through an auditory modality.

48. A method according to claim 46 wherein said stimuli are presented at precise times under computer control.

49. A method according to claim 46 wherein said psychophysiological responses include brain responses.

50. A method according to claim 49 wherein said brain responses include at least one of the following: (a) event-related potentials; and (b) multifaceted electroencephalographic responses.

51. A method according to claim 46 wherein said task involves responding to stimuli through a computer input device.

52. A method according to claim 46 wherein said task performance includes at least one of the following: (a) reaction time; and (b) accuracy.

53. A method for detecting of deception comprising the following steps:
obtaining responses from a subject that contain information regarding a relevant topic through at least one of the following: (a) questioning the subject; and (b) instructing the subject to respond to stimuli;
creating at least one of the following: (a) a set of task instructions to be followed during the process specified in obtaining responses; (b) a specific line of questioning; and (c) a series of one or more stimuli; that elicit the performance of significantly different cognitive tasks by deceptive and truthful subjects respectively;
measuring the psychophysiological manifestations of said cognitive tasks elicited by the procedures specified above; and
analyzing said psychophysiological responses to determine at least one of the following: (a) whether or not the subject is performing the cognitive task that is elicited from a deceptive subject by the procedure specified in the above steps; and (b) whether or not the subject is performing the cognitive task that is elicited from a truthful subject by the procedure specified in the above steps.

54. A method according to claim 53 wherein said at least one of: (a) said set of task instructions; (b) said line of questioning; and (c) said series of stimuli is designed to elicit from a deceptive subject some rehearsed lies and some unrehearsed lies that involve the cognitive task of making up at least some of the information contained in said unrehearsed lies.

55. A method according to claim 54 wherein at least one of: (a) said set of task instructions; (b) said line of questioning; and (c) said series of stimuli comprises a series that progressively increases in at least one of: (a) complexity; (b) scope; (c) detail; (d) divergence from the content of at least some of said unrehearsed lies; and (e) divergence from the central issue of the interrogation being undertaken.

56. A method according to claim 53 wherein said psychophysiological responses include brain responses.

57. A method according to claim 54 wherein said psychophysiological responses include brain responses.

58. A method according to claim 55 wherein said psychophysiological responses include brain responses.

59. A method according to claim 56 wherein said psychophysiological responses include electroencephalographic responses.

60. A method according to claim 57 wherein said psychophysiological responses include electroencephalographic responses.

61. A method according to claim 58 wherein said psychophysiological responses include electroencephalographic responses.

62. A method according to claim 56 wherein said psychophysiological responses include at least one of: (a) event-related brain potentials, (b) multifaceted electroencephalographic response analysis, (c) frequency analysis, and (d) dynamical systems analysis.

63. A method according to claim 57 wherein said psychophysiological responses include at least one of: (a) event-related brain potentials, (b) multifaceted electroencephalographic response analysis, (c) frequency analysis, and (d) dynamical systems analysis.

64. A method according to claim 58 wherein said psychophysiological responses include at least one of: (a) event-related brain potentials, (b) multifaceted electroencephalographic response analysis, (c) frequency analysis, and (d) dynamical systems analysis.
